(19) United States
(12) Patent Application Publication
(10) Pub. No.: US 2025/0152101 A1
(43) Pub. Date: May 15, 2025

(54) INNER RING COMPONENT FOR HUMAN PHYSIOLOGICAL MONITORING RING AND THE HUMAN PHYSIOLOGICAL MONITORING RING

(71) Applicant: LEFY HEALTH TECHNOLOGIES, LLC, Los Angeles, CA (US)

(72) Inventor: Jiangfeng LIN, Shenzhen (CN)

(73) Assignee: LEFY HEALTH TECHNOLOGIES, LLC, Los Angeles, CA (US)

(21) Appl. No.: 19/021,273

(22) Filed: Jan. 15, 2025

Related U.S. Application Data
(63) Continuation of application No. PCT/CN2024/084651, filed on Mar. 29, 2024.

(51) Int. Cl. A61B 5/00; A61B 5/024; A61B 5/1455

(52) U.S. Cl. CPC A61B 5/6826; A61B 5/024; A61B 5/14552; A61B 2560/0462; A61B 2562/0233; A61B 2562/166; A61B 2562/228

(57) ABSTRACT

An inner ring component for a physiological monitoring ring includes an inner ring, a circuit board, and a conduction component. The installation gap between the inner ring and the circuit board accommodates the circuit board having a light source and a light detection component. The light source emits detection light toward the user's finger, and the light detection component receives the reflected light formed by the reflection of the detection light from the user's finger. The conduction component, located in the installation gap, includes a filter and a first light guide that directs the reflected light to the light detection component. Compared to the prior art, the interference from external ambient light to the light detection component can be reduced, thus improving the accuracy of the light detection component's detection of the reflected light, and in turn improving the precision of the physiological information detected by the inner ring component.

### CROSS-REFERENCE TO RELATED APPLICATIONS

This application is a continuation of International Patent Application No. PCT/CN2024/084651 with a filing date of Mar. 29, 2024, designating the United States, now pending. The content of the aforementioned application, including any intervening amendments thereto, is incorporated herein by reference.

### TECHNICAL FIELD

This application relates to the field of wearable devices, and particularly to an inner ring component for a human physiological monitoring ring and a human physiological monitoring ring having the inner ring component.

### BACKGROUND OF THE INVENTION

A human physiological monitoring ring primarily monitors physiological indicators such as blood oxygen and heart rate by wearing the ring on the finger, allowing real-time monitoring of physiological data. In the prior art, the physiological monitoring ring emits detection light through an internal light source and uses a light detection component to receive the reflected light from the finger to monitor physiological data. However, both external ambient light and the reflected light are detected by the light detection component, leading to interference from external light, which reduces the measurement accuracy to the reflected light, thus lowering the precision of the physiological monitoring ring.

### SUMMARY OF THE INVENTION

In order to reduce the impact of external ambient light on the light detection component and improve the accuracy of the physiological monitoring ring, the present application provides an inner ring component for a physiological monitoring ring.

The present application further proposes a physiological monitoring ring.

The inner ring component for the physiological monitoring ring provided in this application adopts the following technical solution:

An inner ring component for a physiological monitoring ring includes: an inner ring; a circuit board, where the circuit board is located within the inner ring, and a mounting gap is defined between the inner ring and the circuit board; the inner ring is adapted to be placed on an outer side of a user's finger, and the circuit board forms a detection circuit; a side of the circuit board near the inner ring is has a light source and a light detection component; the light source is configured to emit detection light toward the user's finger, and the light detection component is configured to receive the reflected light formed by the detection light reflecting off the surface of the user's finger; and the detection circuit is adapted to obtain the user's physiological information based on the detection signal from the light detection component; and a conduction component located within the mounting gap, along a radial direction of the inner ring component, where the conduction component is placed on the outer side of the light detection component and extends toward the inner side of the inner ring component; the conduction component includes a filtering element and a first light guide element; and the filtering element is configured to filter light of a preset wavelength, and the first light guide element is configured to guide the reflected light and the external environmental light after being filtered to the light detection component.

By adopting the above technical solution, the filtering element filters out light of a preset wavelength from the external ambient light, and the first light guide element transmits the reflected light and the filtered external ambient light to the light detection component. Compared to the prior art, this can reduce the interference of external ambient light on the light detection component, thereby improving the accuracy of the reflected light detection by the light detection component, and further enhancing the accuracy of the inner ring component in detecting physiological information.

Preferably, the conduction component comprises at least one filtering element, and the first light guide element is arranged around the light detection component and extends toward the inner side of the inner ring component. The filtering element is located at an end of the first light guide element, which is farthest from the light detection component.

By adopting the above technical solution, where one end of the first light guide element is placed around the light detection component and the filtering element is positioned at the end of the first light guide element, which is farthest from the light detection component, the technical effect of filtering light of a preset wavelength from the external ambient light and guiding the reflected light and the filtered external ambient light to the light detection component can be achieved.

Preferably, the conduction component comprises a plurality of filtering elements, and the plurality of filtering elements are stacked sequentially at the end of the first light guide element that is farthest from the light detection component.

By adopting the above technical solution, designers can choose to use the plurality of filtering elements to filter light of multiple preset wavelengths or to filter light within a preset wavelength range, thus improving the user experience of the inner ring component.

Preferably, the first light guide element defines a first light guiding channel, and the first light guiding channel has a first opening at one end and a second opening at the other end. The light detection component extends into the first opening, and the second opening is positioned opposite to the filtering element.

By adopting the above technical solution, the first light guiding channel can guide the reflected light to the light detection component, which reduces the loss of reflected light during its transmission to the light detection component. This can improve the accuracy of the light detection component in detecting reflected light, and further enhance the accuracy of the inner ring component in detecting physiological information.

Preferably, the conduction component further includes: a second light guide element located on the outer side of the first light guide element along the radial direction of the inner ring component. One end of the second light guide element has an accommodation slot, into which the light source extends. The other end of the second light guide element extends toward the inner side of the inner ring component. The second light guide element is configured to guide the detection light emitted from the light source toward the user's finger.

By adopting the above technical solution, the second light guide element can avoid the detection light directly entering the first light guide element in the mounting gap, thereby minimizing interference to the light detection component from the detection light. This can improve the accuracy of the light detection component in detecting reflected light, further enhancing the accuracy of the inner ring component in detecting physiological information.

Preferably, there is a plurality of light sources, with at least two of the light sources emitting detection light at different frequencies. The light sources are spaced apart along a circumferential direction of the inner ring component.

By adopting the above technical solution, the time interval between the receptions of two reflected light signals by the light detection component can be reduced, thereby increasing the frequency at which the light detection component generates detection signals. This can improve the real-time capability of the inner ring component in detecting physiological information.

Preferably, there are a plurality of light detection components, and the plurality of light detection components are spaced apart along the circumferential direction of the inner ring component. Each light detection component corresponds to at least one light source.

By adopting the above technical solution, designers can arrange each light detection component to correspond to one light source, or arrange each light source to correspond to multiple light detection components, thus enhancing the user experience of the inner ring component.

Preferably, the inner ring is has a clearance hole that runs through the inner ring along the radial direction of the inner ring component. The clearance hole communicates with the mounting gap. The first and second light guide elements both pass through the clearance hole and are adapted to stop against the user's finger.

By adopting the above technical solution, the detection light and the reflected light can be prevented from being blocked by the inner ring, thereby increasing the amount of reflected light received by the light detection component. This improves the accuracy of the detection circuit in obtaining physiological information, further enhancing the accuracy of the inner ring component in detecting physiological information.

Preferably, the inner ring component for the physiological monitoring ring further includes: a sealing element located within the mounting gap and positioned at the clearance hole, and is configured to seal the clearance hole.

By adopting the above technical solution, the sealing element can prevent external liquids from entering the mounting gap, thereby avoiding the possibility of liquids from the external environment entering the circuit board and causing it to malfunction. This can improve the reliability of the inner ring component's operation. It should be noted that the sealing element is made of a high-transparency material, and both the detection light and reflected light can pass through the sealing element.

The present application provides a physiological monitoring ring with the following technical solution:

A physiological monitoring ring, includes: an outer ring; and an inner ring component, where the outer ring is detachably connected to an outer side of the inner ring component along a radial direction of the physiological monitoring ring and the inner ring component is the aforementioned inner ring component for the physiological monitoring ring.

By adopting the above technical solution, the light filtering element in the inner ring component can filter the external ambient light of a preset wavelength, and the first light guide element is configured to guide the reflected light and the filtered external ambient light to the light detection component. Compared with the prior art, this can reduce the interference of external ambient light on the light detection component, thus improving the accuracy of the light detection component in detecting reflected light, and further improving the accuracy of the inner ring component in detecting physiological information.

In summary, the present application includes at least one of the following beneficial technical effects:

By using the filtering element to filter light of a preset wavelength from the external ambient light and guiding the reflected light and filtered external ambient light to the light detection component using the first light guide element, compared with the prior art, it can reduce the interference of external ambient light on the light detection component. This can improve the accuracy of the light detection component in detecting reflected light, and further enhance the accuracy of the inner ring component in detecting physiological information.

Designers can use multiple filtering elements to filter light of multiple preset wavelengths or use multiple filtering elements to filter light within a preset wavelength range, which can improve the user experience of the inner ring component.

It can reduce the time interval between the receptions of two reflected light signals by the light detection component, increase the frequency of detection signal generation by the light detection component, and thus improve the real-time capability of the inner ring component in detecting physiological information.

### BRIEF DESCRIPTION OF THE DRAWINGS

FIG. 1 shows a schematic diagram of an inner ring component according to an embodiment of the present application;

FIG. 2 shows a cross-sectional view of a physiological monitoring ring according to an embodiment of the present application;

FIG. 3 shows a partial enlarged view of the area A shown in FIG. 2;

FIG. 4 shows a cross-sectional view of the physiological monitoring ring from another angle according to the embodiment of the present application;

FIG. 5 shows a partial enlarged view of the area A shown in FIG. 4; and

FIG. 6 shows a schematic diagram of the physiological monitoring ring according to the embodiment of the present application.

### REFERENCE SIGNS DESCRIPTION

100: Inner ring component
200: Physiological monitoring ring
10: Inner ring
101: Mounting gap
102: Clearance hole
20: Circuit board
201: Light source
202: Light detection component
30: Conduction component
301: Filtering element
302: First light guide element
3021: First light guide channel
3022: First opening
3023: Second opening
3024: Emission portion
3025: Incident portion
3026: Conduction portion
303: Second light guide element
3031: Receiving groove
3032: Second light guide channel
3033: Third opening
3034: Fourth opening
40: Sealing member
50: Outer ring
60: Support frame
601: Accommodation cavity

### DETAILED DESCRIPTION OF THE EMBODIMENTS

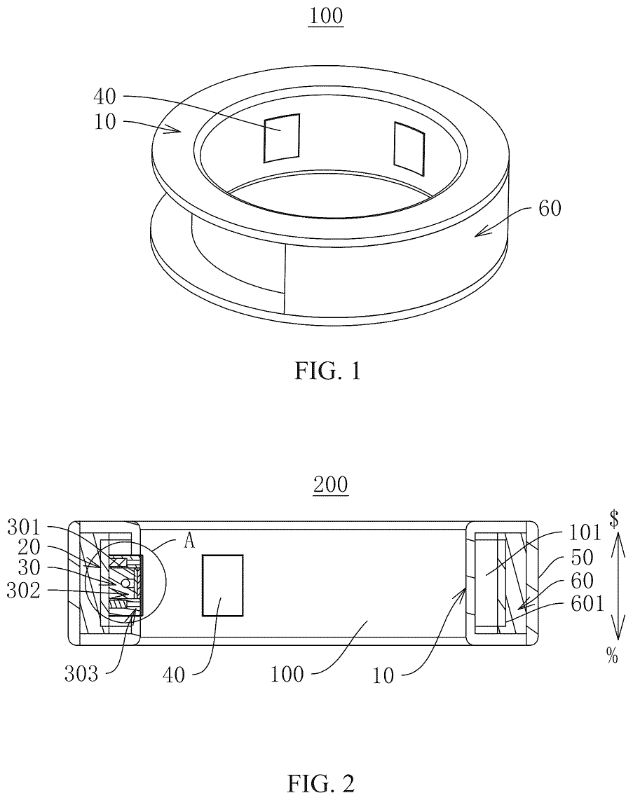

The following provides a more detailed explanation of the present application with reference to FIGS. 1-6.

The embodiment of the present application discloses an inner ring component 100.

Referring to FIGS. 1-3, the inner ring component 100 according to this embodiment includes: an inner ring 10, a circuit board 20, and a conduction component 30. The circuit board 20 is positioned on the outer side of the inner ring 10, and a mounting gap 101 is defined between the inner ring 10 and the circuit board 20. After the circuit board 20 and inner ring 10 are connected together, the parts on the side of the circuit board 20 near the inner ring 10 are located within the mounting gap 101. This design avoids interference between the parts on the side of the circuit board 20 near the inner ring 10 and the inner ring 10, which can minimize the likelihood of failure of the circuit board 20, thus improving the operational reliability of the inner ring component 100. Additionally, the circuit board 20 and the inner ring 10 can be fixed together by glue filling. After the glue filling is completed, the mounting gap 101 is filled with glue, and the side of the circuit board 20 farthest from the inner ring 10 is covered with glue. It is important to note that the circuit board 20 is an annular flexible circuit board and extends along the circumferential direction of the inner ring component 100.

In some specific embodiments, the material of the inner ring 10 may be ceramic; however, the present application is not limited to this, and materials such as titanium or copper may also be used for the inner ring 10.

Furthermore, the inner ring component 100 may also include a support frame 60. The side of the support frame 60 near the inner ring 10 defines an accommodation cavity 601, within which the circuit board 20 is placed. The support frame 60, with the circuit board 20 inside the accommodation cavity 601, is then positioned on the outer side of the inner ring 10. In assembling the inner ring component 100, compared to directly fixing the circuit board 20 to the outer side of the inner ring 10, this arrangement can reduce the assembly difficulty between the circuit board 20 and the inner ring 10, thus improving the manufacturing efficiency of the inner ring component 100. It should be noted that the support frame 60 is a flexible support frame 60.

The inner ring 10 is adapted to be placed on the outer side of the user's finger, and the circuit board 20 is formed with a detection circuit. On the side of the circuit board 20 close to the inner ring 10, a light source 201 and a light detection component 202 are positioned. Both the light source 201 and the light detection component 202 are located within the mounting gap 101. The light source 201 is configured to emit detection light toward the user's finger, and the light detection component 202 is configured to receive the reflected light formed by the reflection of the detection light from the user's finger surface. The detection circuit is adapted to obtain the user's physiological information based on the detection signal from the light detection component 202. Both the light source 201 and the light detection component 202 are communicatively connected to the detection circuit.

In some specific embodiments, the light source 201 can be a light-emitting diode (LED), and the light detection component 202 can be a photodiode. However, the present application is not limited to these options. The light source 201 can also be a laser diode, and the light detection component 202 can be a photoconductor, etc.

Specifically, the light source 201 emits the detection light, which passes through the inner ring 10 and makes contact with the user's finger. A portion of the detection light is absorbed by the user's finger, while the other portion is reflected by the user's finger, forming the reflected light. The reflected light passes through the inner ring 10 and is received by the light detection component 202, generating a detection signal. The detection circuit uses the detection signal from the light detection component 202 to obtain the user's physiological information. This process enables the acquisition of the user's physiological information using the light source 201, the light detection component 202, and the detection circuit.

Moreover, by setting the light source 201 to different light-emitting diodes, the light source 201 can emit different detection light beams. These different detection light beams can be configured to detect different physiological information of the human body. In some specific embodiments, when the light-emitting diode is an infrared LED, the detection light can be configured to detect blood oxygen saturation. When the light-emitting diode is a green LED, the detection light can be configured to detect heart rate. Designers can set the light source 201 to the corresponding LED according to the user's needs, thereby improving the user experience of the inner ring component 100.

The conduction component 30 is positioned within the mounting gap 101 and extends along the radial direction of the inner ring component 100. One end of the conduction component 30 is placed on the outside of the light detection component 202, while the other end extends toward the inner side of the inner ring component 100. The conduction component 30 includes a filtering element 301 and a first light guide element 302. The filtering element 301 is configured to filter light of a preset wavelength, and the first light guide element 302 is configured to guide the reflected light to the light detection component 202.

It should be noted that the preset wavelength refers to wavelengths outside the wavelength of the detection light emitted by the light source 201. In other words, the preset wavelength does not include the wavelength of the detection light. The preset wavelength can either be a single wavelength value or a range of wavelength values.

In some specific embodiments, when the wavelength of the detection light emitted by the light source 201 is a specific wavelength value A, the preset wavelength is a wavelength range that does not include the specific wavelength value A. When the wavelength of the detection light emitted by the light source 201 is a specific wavelength range B, the preset wavelength is a wavelength range that does not include the specific wavelength range B. Specifically, when the wavelength range of the detection light emitted by the light source 201 is 500 nm to 600 nm, the preset wavelength is a wavelength range that does not include 500 nm to 600 nm.

Specifically, when the detection light is reflected by the user's finger and forms reflected light, both the reflected light and external ambient light will enter the conduction component 30. The filtering element 301 filters the preset wavelength light from the external ambient light, and the first light guide element 302 conducts the reflected light and the filtered external ambient light to the light detection component 202, thus reducing the interference of external ambient light on the light detection component 202.

Additionally, in other specific embodiments, along the radial direction of the inner ring component 100, the sidewall of the inner ring 10 that is adapted to contact the user's finger is provided with a transparent section opposite the circuit board 20. The detection light passes through this transparent section to contact the user's finger, and the reflected light passes through the transparent section into the first light guide element 302. The first light guide element 302 then conducts the reflected light to the light detection component 202. This arrangement allows the light detection component 202 to be positioned on the circuit board 20 in a location not opposite to the transparent section, which can minimize the direct reception of external ambient light by the light detection component 202, thus improving the accuracy of the inner ring component 100 in detecting the user's physiological information.

Furthermore, the inner ring component 100 also includes a battery and a wireless charging coil. The battery is electrically connected to both the circuit board 20 and the wireless charging coil. The battery is configured to power the light source 201 and the light detection component 202 on the circuit board 20, while the wireless charging coil is configured to charge the battery.

Thus, by using the filtering element 301 to filter the preset wavelength light from the external ambient light, and utilizing the first light guide element 302 to conduct the reflected light and the filtered external ambient light to the light detection component 202, compared to the prior art, the interference of external ambient light on the light detection component 202 is reduced. This can enhance the accuracy of the light detection component 202 in detecting the reflected light and, consequently, improve the precision of the inner ring component 100 in detecting the user's physiological information.

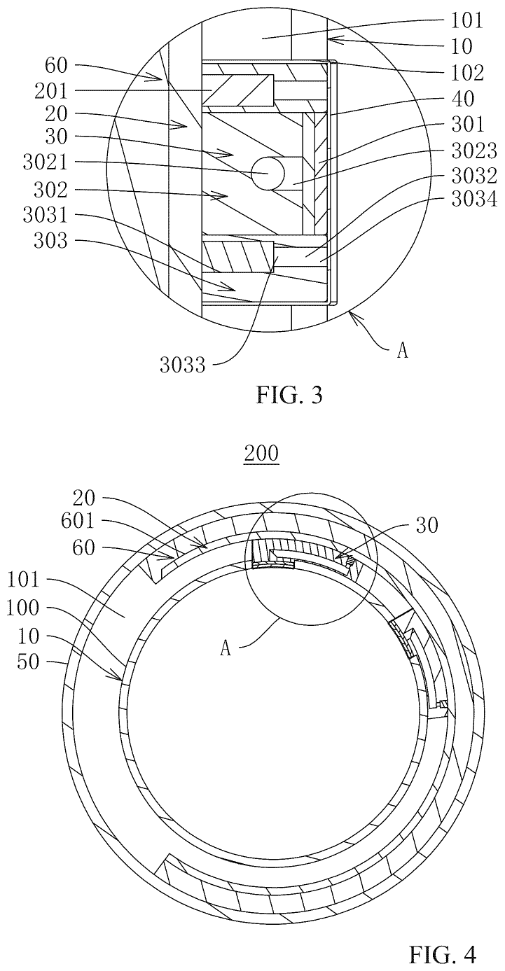

With reference to FIGS. 3-5, in some embodiments of this application, the filtering element 301 is at least one. When the filtering element 301 filters light of a single preset wavelength, the filtering element 301 can be a single element. When the filtering element 301 filters light of a preset wavelength range, the filtering element 301 can be one or more elements. In some specific embodiments, the filtering element 301 can be a filtering plate, but the application is not limited to this. The filtering element 301 can also be a bandpass filter, low-pass filter, or high-pass filter, among others. It should be noted that when the filtering element 301 is a filtering plate, it can filter light of a single preset wavelength value. When the filtering element 301 is a bandpass filter and/or low-pass filter and/or high-pass filter, it can filter light of a preset wavelength range. Designers can select the filtering element 301 based on actual usage requirements, using a filtering plate and/or bandpass filter and/or low-pass filter and/or high-pass filter, thereby improving the user experience of the inner ring component 100.

Furthermore, one end of the first light guide element 302 is set around the outside of the light detection component 202, and the other end extends toward the inner side of the inner ring component 100. Specifically, the end of the first light guide element 302 set around the light detection component 202 is positioned against the side of the circuit board 20 that is near the inner ring 10. This means that the light detection component 202 is entirely located inside the first light guide element 302. In some specific embodiments, when the sidewall of the inner ring 10, which is adapted to contact the user's finger, is provided with a transparent section, the end of the first light guide element 302, which is far from the light detection component 202, is positioned against the side of the transparent section that is close to the circuit board 20. The reflected light passes through the transparent section and enters the first light guide element 302, which then conducts the reflected light to the light detection component 202.

In other specific embodiments, when the sidewall of the inner ring 10, adapted to contact the user's finger, is provided with a clearance hole 102, the end of the first light guide element 302, which is far from the light detection component 202, passes through the clearance hole 102 and is adapted to contact the user's finger. The reflected light directly enters the first light guide element 302, which then conducts the reflected light to the light detection component 202.

Furthermore, in some embodiments of this application, when the light detection component 202 is not opposite the transparent section or the clearance hole 102 of the inner ring 10, one end of the first light guide element 302 close to the light detection component 202 is configured as an emission portion 3024, and the other end of the first light guide element 302 close to the inner ring 10 is configured as an incident portion 3025. The first light guide element 302 also includes a conduction portion 3026, which communicates between the incident portion 3025 and the emission portion 3024. The conduction portion 3026 is configured to conduct the light located in the incident portion 3025 to the emission portion 3024. Specifically, the reflected light enters the first light guide element 302 from the incident portion 3025, then is conducted by the conduction portion 3026 to the emission portion 3024, and finally exits from the emission portion 3024 to be received by the light detection component 202.

In other embodiments of this application, when the light detection component 202 is opposite the transparent section or the clearance hole 102 of the inner ring 10, the reflected light is conducted by the first light guide element 302 to the light detection component 202.

The filtering element 301 can be arranged at one end of the first light guide element 302, which is far from the light detection component 202. When both the reflected light and external environmental light enter the first light guide element 302, the filtering element 301 filters light of a preset wavelength from the external environmental light. This can reduce the interference of external environmental light on the light detection component 202, thereby improving the accuracy of the light detection component 202 in detecting the reflected light, which further helps to improve the precision of the inner ring component 100 in detecting physiological information.

Moreover, in other specific embodiments, the filtering element 301 can also be arranged at the end of the first light guide element 302 that is close to the light detection component 202, or both the end of the first light guide element 302 far from the light detection component 202 and the end of the filtering element 301 close to the light detection component 202 can each be provided with a filtering element 301. Designers can place the filtering element 301 at different positions along the first light guide element 302 according to actual usage requirements, thus enhancing the user experience of the inner ring component 100.

By setting one end of the first light guide element 302 around the light detection component 202, and placing the filtering element 301 at the end of the first light guide element 302 that is far from the light detection component 202, it is possible to filter light of a preset wavelength from the external environmental light and conduct both the reflected light and the filtered external environmental light to the light detection component 202, thereby achieving the technical effect of improving the accuracy of the physiological information detection by the inner ring component 100.

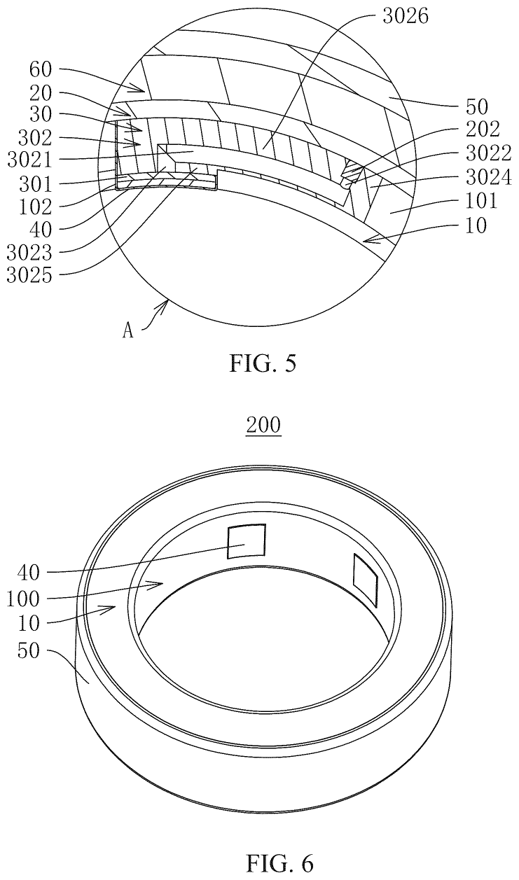

Referring to FIGS. 3 and 5, in some embodiments of this application, the filtering elements 301 are multiple, with the multiple filtering elements 301 stacked sequentially at the far end of the first light guide element 302, away from the light detection component 202. The multiple filtering elements 301 filter multiple single preset wavelength values or filter a range of preset wavelength values. Designers can use multiple filtering elements 301 to filter multiple single preset wavelengths or to filter a range of preset wavelengths according to the usage requirements, thus enhancing the user experience of the inner ring component 100.

In the embodiment shown in FIG. 4, there are two filtering elements 301. One of the filtering elements 301 is a low-pass filter, and the other is a high-pass filter. The two filtering elements 301 together filter light from the external environment in a preset wavelength range.

Referring to FIG. 5, in some embodiments of this application, the first light guide element 302 defines a first light guide channel 3021, and the first light guide channel 3021 forms a first opening 3022 at one end and a second opening 3023 at the other end. The light detection component 202 extends into the first opening 3022, and the second opening 3023 is opposite the filtering element 301. The reflected light passes sequentially through the filtering element 301, the second opening 3023, the first light guide channel 3021, and the second opening 3023 before being received by the light detection component 202.

Additionally, in some embodiments of this application, when the light detection component 202 is not opposite the transparent section or clearance hole 102 of the inner ring 10, the first opening 3022 is located at the emission portion 3024, and the second opening 3023 is located at the incident portion 3025. The first light guide channel 3021 is located at the conduction portion 3026.

It is noteworthy that the first light guide element 302 uses optical waveguides to conduct the reflected light to the light detection component 202. The inner layer of the first light guide channel 3021, configured to conduct the reflected light, is made from a high refractive index material, while the outer layer of the inner layer is coated with a low refractive index material. The first light guide channel 3021 conducts the reflected light via the total internal reflection effect, and during the process of the reflected light being conducted to the light detection component 202, this total internal reflection effect helps reduce the loss of reflected light.

Through the use of the first light guide channel 3021 to conduct the reflected light to the light detection component 202, the loss of the reflected light during its conduction to the light detection component 202 can be minimized, thereby improving the accuracy of the light detection component 202 in detecting the reflected light. This, in turn, can further enhance the accuracy of the inner ring component 100 in detecting physiological information from the human body.

Referring to FIGS. 2 and 3, in some embodiments of this application, the conduction component 30 may further include a second light guide element 303. The second light guide element 303 is disposed on the outer side of the first light guide element 302, and the outer wall of the second light guide element 303 can either be in contact with the outer wall of the first light guide element 302, or there can be a gap between the outer wall of the second light guide element 303 and the outer wall of the first light guide element 302. Specifically, in the embodiment shown in FIG. 4, the outer wall of the second light guide element 303 is in contact with the outer wall of the first light guide element 302. Along the height direction of the inner ring component 100 (which refers to the vertical direction in FIG. 2), the second light guide element 303 can be positioned either above or below the first light guide element 302.

Additionally, in the radial direction of the inner ring component 100, the end of the second light guide element 303 near the circuit board 20 has a receiving groove 3031, into which the light source 201 is completely inserted. In other words, the end of the second light guide element 303 near the circuit board 20 is in contact with the circuit board 20, and the other end of the second light guide element 303 extends toward the inner side of the inner ring component 100. When the inner ring 10 has a transparent portion, the far end of the second light guide element 303 (the one away from the circuit board 20) is in contact with the transparent portion. When the inner ring 10 has a clearance hole 102, the far end of the second light guide element 303 passes through the clearance hole 102 and is designed to be in contact with the user's finger.

Furthermore, the second light guide element 303 is made of an opaque material. The second light guide element 303 is configured to conduct the detection light emitted by the light source 201 toward the user's finger. Specifically, the second light guide element 303 defines a second light guide channel 3032, which forms a third opening 3033 at one end and a fourth opening 3034 at the other end. The light source 201 is inserted into the third opening 3033, and the fourth opening 3034 is positioned opposite the user's finger. When the inner ring 10 has a transparent portion, the fourth opening 3034 is aligned with the transparent portion of the inner ring 10 and the user's finger. The detection light emitted by the light source 201 passes sequentially through the third opening 3033, the second light guide channel 3032, the fourth opening 3034, and the inner ring 10 to contact the user's finger.

When the inner ring 10 has a clearance hole 102, the fourth opening 3034 is positioned opposite the user's finger, and the detection light emitted by the light source 201 passes sequentially through the third opening 3033, the second light guide channel 3032, and the fourth opening 3034 to contact the user's finger.

Additionally, the second light guide element 303 helps to prevent the detection light from directly entering the first light guide element 302 within the installation gap 101, which minimizes the interference of the detection light on the light detection component 202. This, in turn, improves the accuracy of the light detection component 202 in detecting the reflected light, thereby enhancing the accuracy of the inner ring component 100 in detecting physiological information from the human body.

Furthermore, the second light guide element 303 and the inner ring 10 can be constructed as a single piece, meaning that the side of the inner ring 10 near the circuit board has the same structure as the second light guide element 303.

Referring to FIGS. 2 to 4, in some embodiments of this application, the light source 201 consists of multiple sources, with any two adjacent light sources 201 spaced apart along the circumferential direction of the inner ring component 100. Additionally, in the embodiment shown in FIG. 4, multiple light sources 201 can also be spaced apart along the height direction of the inner ring component 100. The detection light emitted by at least two light sources 201 has different frequencies, meaning that at least two light sources 201 emit detection light toward the user's finger at different time intervals, forming reflected light with different time intervals. This reduces the time interval between two reflected light signals received by the light detection component 202, increases the frequency at which the light detection component 202 generates detection signals, and thus improves the real-time performance of the inner ring component 100 in detecting physiological information from the human body.

Furthermore, the detection light emitted by at least two light sources 201 is different, and the different detection light is absorbed and reflected by the human finger, forming different reflected light signals. The light detection component 202 receives the different reflected light signals and generates corresponding detection signals. The detection circuit acquires different physiological information from the user based on the different detection signals generated by the light detection component 202, thereby enhancing the user experience of the inner ring component 100. In some specific embodiments, the detection light may be infrared light or green light. Infrared light is configured to detect the user's blood oxygen level, while green light is configured to detect the user's heart rate.

Furthermore, the second light guide element 303 consists of multiple elements, and the multiple second light guide elements 303 are spaced apart along the circumferential direction of the inner ring component 100 and/or along the height direction of the inner ring component 100. The multiple second light guide elements 303 correspond one-to-one with the multiple light sources 201.

Referring to FIGS. 4 and 5, in some embodiments of this application, the light detection component 202 consists of multiple components, which are spaced apart along the circumferential direction of the inner ring component 100. Each light detection component 202 corresponds to at least one light source 201. That is, each light detection component 202 may correspond to one light source 201, or each light detection component 202 may correspond to multiple light sources 201.

When each light detection component 202 corresponds to a single light source 201, the light detection component 202 only receives the reflected light formed by the detection light emitted by the corresponding light source 201. This minimizes interference from reflected light formed by detection light emitted by light sources 201 that do not correspond to the light detection component 202, thus improving the accuracy of the light detection component 202 in detecting reflected light.

When each light detection component 202 corresponds to multiple light sources 201, that is, multiple light detection components 202 can receive the reflected light formed by detection light emitted by multiple light sources 201. This ensures that the reflected light formed by detection light from multiple light sources 201 is effectively received by the light detection component 202, thereby improving the accuracy of the light detection component 202 in detecting reflected light and further enhancing the accuracy of the inner ring component 100 in detecting the user's physiological information.

Designers can choose to have each light detection component 202 correspond to one light source 201, or each light detection component 202 correspond to multiple light sources 201, depending on the design requirements, thereby improving the user experience of the inner ring component 100.

Furthermore, both the optical filter 301 and the first light guide component 302 are multiple. The multiple optical filters 301 and the multiple first light guide components 302 are spaced apart along the circumferential direction of the inner ring component 100. The multiple optical filters 301 and the multiple first light guide components 302 are arranged one-to-one with multiple light detection components 202.

Referring to FIGS. 3 to 5, in some embodiments of this application, the sidewall of the inner ring 10, which is intended to contact the user's finger, is provided with a clearance hole 102 that passes through the inner ring 10 in the radial direction of the inner ring component 100. The clearance hole 102 communicates with the installation gap 101. Both the first light guide component 302 and the second light guide element 303 pass through the clearance hole 102 and are adapted to contact the user's finger. Compared to the detection light passing through the inner ring 10 and contacting the user's finger, and the reflected light passing through the inner ring 10 to be received by the light detection component 202, this arrangement can avoid the detection light and reflected light from being blocked by the inner ring 10, thereby increasing the amount of reflected light that the light detection component 202 can receive. This enhances the accuracy of the detection circuit in obtaining the user's physiological information, which further improves the precision of the inner ring component 100 in detecting physiological information.

Furthermore, when at least one of the first light guide component 302 and the second light guide element 303 is multiple, the clearance hole 102 is also multiple. The multiple clearance holes 102 are spaced apart along the circumferential direction of the inner ring component 100, with the multiple clearance holes 102 arranged one-to-one with the multiple first light guide components 302 and/or the multiple second light guide elements 303.

Additionally, the end of the first light guide component 302, which is intended to contact the user's finger, and the end of the second light guide element 303, which is intended to contact the user's finger, are both flush with the inner side of the inner ring 10. This configuration can prevent interference between at least one of the first light guide component 302 and the second light guide element 303 and the user's finger, ensuring that the user can comfortably wear the inner ring component 100.

Referring to FIGS. 3 and 5, in some embodiments of this application, the inner ring assembly 100 further includes: a sealing member 40. The sealing member 40 is disposed within the installation gap 101. One end of the sealing member 40 is positioned at the clearance hole 102 and is configured to seal the clearance hole 102, while the other end is fitted around the outer sides of the first light guide component 302 and the second light guide element 303. This arrangement helps prevent liquids from the external environment from entering the installation gap 101, thereby avoiding the possibility of liquids entering the circuit board 20 and causing the circuit board 20 to malfunction. This can enhance the reliability of the inner ring assembly 100. It should be noted that the sealing member 40 is made of a high-transparency material, and both the detection light and the reflected light can pass through the sealing member 40.

Further, the sealing member 40 can be made by a gel-filling method. When the circuit board 20 and the inner ring 10 are fixed together by the gel-filling method, the gel configured to fix them also seals the clearance hole 102 and solidifies to form the sealing member 40. It should be noted that the gel used is transparent, allowing both the detection light and the reflected light to pass through the gel.

Based on this, the application also further discloses a physiological monitoring ring 200 for the human body. Referring to FIG. 6, according to the embodiment of this application, the physiological monitoring ring 200 includes: an outer ring 50 and the inner ring assembly 100. Along the radial direction of the physiological monitoring ring 200, the outer ring 50 is detachably connected to the outer side of the inner ring assembly 100. The inner ring assembly 100 is used for monitoring human physiological information and is the same as the inner ring assembly 100 described in the above embodiments. In some specific embodiments, the outer ring 50 and the inner ring assembly 100 can be connected by adhesive, and after heating the physiological monitoring ring 200, the outer ring 50 and the inner ring assembly 100 can be separated. The material of the outer ring 50 may be gold, but this is not limited, and the outer ring 50 can also be made of materials like titanium or transparent resin.

According to the embodiment of the physiological monitoring ring 200 described in this application, the outer ring 50 is detachably connected to the outer side of the inner ring assembly 100. By using the optical filter 301 in the inner ring assembly 100 to filter out light of preset wavelengths from the external environment, and by using the first light guide component 302 to guide the reflected light and the filtered external light to the light detection component 202, compared to the prior art, the interference of external environmental light on the light detection component 202 can be reduced. This improves the accuracy of the light detection component 202 in detecting the reflected light, which in turn enhances the accuracy of the inner ring assembly 100 in monitoring human physiological information.

The above are preferred embodiments of this application, and are not intended to limit the scope of protection. Therefore, any equivalent changes made based on the structure, shape, and principles described in this application should be included within the protection scope of this application.

What is claimed is:

1. An inner ring component for a human physiological monitoring ring, comprising:
an inner ring (10);
a circuit board (20), wherein the circuit board (20) is disposed on the inner ring (10), and an installation gap (101) is defined between the inner ring (10) and the circuit board (20); the inner ring (10) is designed to be worn on an outer side of a user's finger, and the circuit board (20) forms a detection circuit; a side of the circuit board (20) near the inner ring (10) has a light source (201) and a light detection component (202), the light source (201) emits detection light toward the user's finger, and the light detection component (202) receives the reflected light formed by the detection light reflected from the user's finger; the detection circuit is configured to obtain the user's physiological information based on a signal detected by the light detection component (202); and
a conduction component (30), partially located in the installation gap (101); wherein in a radial direction of the inner ring component (100), the conduction component (30) is positioned outside the light detection component (202) and extends inwardly toward the inner ring component (100); and the conduction component (30) includes:
a first light guide element (302 configured to conduct the reflected light to the light detection component (202).

2. The inner ring component according to claim 1, wherein the conduction component (30) further includes at least one filtering element (301), and the first light guide element (302) is disposed outside the light detection component (202) and extends inwardly toward the inner ring component (100), with the filtering element (301) placed at a far end of the first light guide element (302) away from the light detection component (202).

3. The inner ring component according to claim 1, wherein the conduction component (30) further comprises a plurality of filtering elements (301), with the filtering elements (301) stacked in sequence at a far end of the first light guide element (302) away from the light detection component (202).

4. The inner ring component according to claim 2, wherein the first light guide element (302) defines a first light guide channel (3021), with the first light guide channel (3021) having a first opening (3022) at one end and a second opening (3023) at another end; the light detection component (202) extends into the first opening (3022), and the second opening (3023) is opposite to the filtering element (301).

5. The inner ring component according to claim 2, wherein the conduction component (30) further includes a second light guide element (303) located outside the first light guide element (302); in the radial direction of the inner ring component (100), the second light guide element (303) has a receiving groove (3031) at one end, with the light source (201) extending into the receiving groove (3031); another end of the second light guide element (303) extends inwardly toward the inner ring component (100) and is configured to conduct the detection light emitted by the light source (201) toward the user's finger.

6. The inner ring component according to claim 5, wherein there are a plurality of light sources (201), with at least two light sources (201) emitting detection light at different frequencies, with adjacent light sources (201) being spaced apart along a circumferential direction of the inner ring component (100).

7. The inner ring component according to claim 1, wherein there are a plurality of light detection components (202), with the plurality of light detection components (202) spaced apart along a circumferential direction of the inner ring component (100), with each of the plurality of light detection components (202) corresponding to at least one light source (201).

8. The inner ring component according to claim 5, wherein the inner ring (10) has a clearance hole (102) passing through the inner ring (10) along the radial direction of the inner ring component (100), with the clearance hole (102) communicating with the installation gap (101); the first light guide element (302) and the second light guide element (303) both pass through the clearance hole (102) and are designed to contact the user's finger.

9. The inner ring component according to claim 8, further including a sealing member (40) disposed in the installation gap (101) and at the clearance hole (102) for sealing the clearance hole (102).

10. The inner ring component according to claim 6, wherein there are a plurality of light detection components (202), with the plurality of light detection components (202) spaced apart along a circumferential direction of the inner ring component (100), with each of the plurality of light detection components (202) corresponding to at least one light source (201).

11. A human physiological monitoring ring, including:
an outer ring (50); and
an inner ring component (100), wherein the outer ring (50) is detachably connected to an outer side of the inner ring component (100) along a radial direction of the human physiological monitoring ring (200); and the inner ring component (100) is as described in claim 1.
